(19) United States
(12) Patent Application Publication
(10) Pub. No.: US 2026/0234715 A1
(43) Pub. Date: Aug. 13, 2026

(54) METHODS AND SYSTEMS FOR MODELING INFERENCE OF MUTATION IMPACT

(71) Applicant: GeneDx, LLC, Gaithersburg, MD (US)

(72) Inventors: Vincent Ustach, Baltimore, MD (US); Timothy Laurent, San Francisco, CA (US); Amber Begtrup, Cincinnati, OH (US); Maria J. Guillen Sacoto, Natick, MA (US); Kathleen S. Hruska, Baltimore, MD (US); Hákon Guðbjartsson, Reykjavík (IS)

(21) Appl. No.: 19/185,003

(22) Filed: Apr. 21, 2025

Related U.S. Application Data
(60) Provisional application No. 63/756,944, filed on Feb. 11, 2025.

(51) Int. Cl. C12Q 1/6869

(52) U.S. Cl. CPC C12Q 1/6869

(57) ABSTRACT

Provided herein are systems, methods, computer-readable media, and techniques for analyzing variants, including: (A) obtaining assay data corresponding to an assay for a subject; (B) identifying a plurality of variants in said assay data, wherein said plurality of variants comprise at least about one thousand variants; and (C) selecting a subset of variants from said plurality of variants, wherein said subset of variants comprises a variant most relevant to diagnosing said subject.

### CROSS-REFERENCE

This application claims the benefit of U.S. Provisional Application No. 63/756,944 filed Feb. 11, 2025, which is incorporated by reference herein in its entirety.

### BACKGROUND

Diagnostic genetic testing may involve examining a subject's DNA to detect genetic mutations that may cause illness or disease via sifting through thousands of detected variants in assay data collected from the subject. Diagnostic genetic testing may play a fundamental role in determining the subject's risk of developing certain diseases and uncovering genetic causes of illnesses that the subject may be suffering from.

### SUMMARY

The present disclosure provides systems, methods, computer-readable media, and techniques for a Model for Inference of Mutation Impact (MIMI) Variant Ranker that leverages machine learning to rank the relevance of variants within an exome-based test or genome-based test, to help identify a diagnostic variant (e.g., a variant (or variants) most relevant to diagnosing a subject or patient). In improving the variant review process, at least in part by automating the variant scoring process, genetic experts may focus their review of assay data from subjects (e.g., patients), improving their efficiency, decision-making, and diagnostic accuracy.

In an aspect, the present disclosure provides a computer system for analyzing variants, comprising: one or more processors; and one or more memories storing computer-executable instructions that, when executed, cause the one or more processors to: (a) obtain assay data corresponding to an assay for a subject; (b) process said assay data to identify a plurality of variants in said assay data, wherein said plurality of variants comprise at least about one thousand variants; and (c) select a subset of variants from said plurality of variants, wherein said subset of variants comprises a diagnostic variant, wherein said selecting has an accuracy rate of at least about 90%.

In some embodiments, selecting said subset of variants in (c) further comprises generating, using a machine learning model, a score for each variant of said plurality of variants, wherein said score for each variant of said plurality of corresponds to a relevance of said variant to diagnosing said subject. In some embodiments, selecting said subset of variants in (c) further comprises ranking said plurality of variants in a ranked order, based at least in part on said score for each variant of said plurality of variants. In some embodiments, said plurality of variants comprise at least about three thousand variants. In some embodiments, said plurality of variants comprises at least about ten thousand variants.

In some embodiments, selecting said subset of variants in (c) further comprises using a trained machine learning model that comprises one or more decision trees. In some embodiments, said trained machine learning model comprises a random forest. In some embodiments, said assay comprises one or both of an exome-based test or a genome-based test. In some embodiments, said subset of variants comprises at most about one hundred variants of said plurality of variants, and wherein said accuracy rate is at least about 99.9%. In some embodiments, said subset of variants comprises at most about twenty variants of said plurality of variants, and wherein said accuracy rate is at least about 99.9%.

In some embodiments, said subset of variants comprises at most about ten variants of said plurality of variants, and wherein said accuracy rate is at least about 99.7%. In some embodiments, said subset of variants comprises at most about five variants of said plurality of variants, and wherein said accuracy rate is at least about 99%. In some embodiments, said subset of variants comprises at most about three variants of said plurality of variants, and wherein said accuracy rate is at least about 98%.

In some embodiments, the one or more processors of the computer system are further configured to (d) cause a display to present said subset of variants to a user. In some embodiments, said user is one or both of a healthcare provider of said subject or a geneticist. In some embodiments, said subject is suspected of or is diagnosed with a Mendelian disease. In some embodiments, said diagnostic variant is a variant most relevant to diagnosing said subject.

In another aspect, the present disclosure provides a method for analyzing a plurality of variants, having at least about one thousand variants, collected in an assay from a subject, comprising selecting, from said plurality of variants, a subset of variants comprising a diagnostic variant, wherein said subset of variants comprises at most about ten variants of said plurality of variants, wherein said selecting has an accuracy rate of at least about 90%.

In yet another aspect, the present disclosure provides a method for analyzing variants, comprising: (a) obtaining assay data corresponding to an assay for a subject; (b) identifying a plurality of variants in said assay data, wherein said plurality of variants comprise at least about one thousand variants; and (c) selecting a subset of variants from said plurality of variants, wherein said subset of variants comprises a diagnostic variant, wherein said selecting has an accuracy rate of at least about 90%.

In an aspect, the present disclosure provides a computer system for analyzing variants, comprising: one or more processors; and one or more memories storing computer-executable instructions that, when executed, cause the one or more processors to: (a) obtain assay data corresponding to an assay for a subject; (b) process the assay data to identify a plurality of variants in the assay data, wherein the plurality of variants comprise at least about one thousand variants; and (c) select a subset of variants from the plurality of variants, wherein the subset of variants comprises a diagnostic variant, wherein the selecting has an accuracy rate of at least about 90%.

In some embodiments, selecting the subset of variants in (c) further comprises: generating, using a machine learning model, a score for each variant of the plurality of variants, wherein the score for each variant of the plurality of corresponds to a relevance of the variant to diagnosing the subject. In some embodiments, selecting the subset of variants in (c) further comprises: ranking the plurality of variants in a ranked order, based at least in part on the score for each variant of the plurality of variants.

In some embodiments, the plurality of variants comprise at least about three thousand variants. In some embodiments, the plurality of variants comprises at least about ten thousand variants.

In some embodiments, selecting the subset of variants in (c) further comprises using a machine learning model that comprises one or more decision trees. In some embodiments, the machine learning model comprise a random forest. In some embodiments, the machine learning model is trained.

In some embodiments, the assay comprises one or both of an exome-based test or a genome-based test.

In some embodiments, the subset of variants comprises at most about one hundred variants of the plurality of variants, and wherein the accuracy rate is at least about 99.9%. In some embodiments, the subset of variants comprises at most about twenty variants of the plurality of variants, and wherein the accuracy rate is at least about 99.9%. In some embodiments, the subset of variants comprises at most about ten variants of the plurality of variants, and wherein the accuracy rate is at least about 99.7%. In some embodiments, the subset of variants comprises at most about five variants of the plurality of variants, and wherein the accuracy rate is at least about 99%. In some embodiments, the subset of variants comprises at most about three variants of the plurality of variants, and wherein the accuracy rate is at least about 98%.

In some embodiments, the method further comprises: (d) causing a display to present the subset of variants to a user. In some embodiments, the user is one or both of a healthcare provider of the subject or a geneticist.

In some embodiments, the subject is suspected of or is diagnosed with a Mendelian disease.

In some embodiments, the diagnostic variant is a variant most relevant to diagnosing the subject.

In another aspect, the present disclosure provides a computer system for analyzing a plurality of variants, having at least about one thousand variants, collected in an assay from a subject, comprising one or more processors; and one or more memories storing computer-executable instructions that, when executed, cause the one or more processors to: select, from the plurality of variants, a subset of variants comprising a diagnostic variant, wherein the subset of variants comprises at most about ten variants of the plurality of variants, wherein the selecting has an accuracy rate of at least about 90%.

In another aspect, the present disclosure provides a method for analyzing a plurality of variants, having at least about one thousand variants, collected in an assay from a subject, comprising selecting, from the plurality of variants, a subset of variants comprising a diagnostic variant, wherein the subset of variants comprises at most about ten variants of the plurality of variants, wherein the selecting has an accuracy rate of at least about 90%.

In some embodiments, selecting the subset of variants further comprises: generating, using a machine learning model, a score for each variant of the plurality of variants, wherein the score for each variant of the plurality of corresponds to a relevance of the variant to diagnosing the subject.

In some embodiments, selecting the subset of variants further comprises: ranking the plurality of variants in a ranked order, based at least in part on the score for each variant of the plurality of variants.

In some embodiments, the plurality of variants comprises at least about three thousand variants. In some embodiments, the plurality of variants comprises at least about ten thousand variants.

In some embodiments, selecting the subset of variants further comprising using a machine learning model that comprises one or more decision trees. In some embodiments, the machine learning model comprises a random forest. In some embodiments, the machine learning model is trained.

In some embodiments, the assay comprises one or both of an exome-based test or a genome-based test.

In some embodiments, the subset of variants comprises at most about one hundred variants of the plurality of variants, and wherein the accuracy rate is at least about 99.9%. In some embodiments, the subset of variants comprises at most about twenty variants of the plurality of variants, and wherein the accuracy rate is at least about 99.9%. In some embodiments, the subset of variants comprises at most about ten variants of the plurality of variants, and wherein the accuracy rate is at least about 99.7%. In some embodiments, the subset of variants comprises at most about five variants of the plurality of variants, and wherein the accuracy rate is at least about 99%. In some embodiments, the subset of variants comprises at most about three variants of the plurality of variants, and wherein the accuracy rate is at least about 98%.

In some embodiments, the method further comprises: causing a display to present the subset of variants to a user.

In some embodiments, the user is one or both of a healthcare provider of the subject or a geneticist.

In some embodiments, the subject is suspected of or is diagnosed with a Mendelian disease. In some embodiments, the diagnostic variant is a variant most relevant to diagnosing the subject.

Another aspect of the present disclosure provides a non-transitory computer readable medium comprising machine executable code that, upon execution by one or more computer processors, implements any of the methods or techniques above or elsewhere herein.

Another aspect of the present disclosure provides a system comprising one or more computer processors and computer memory coupled thereto. The computer memory comprises machine executable code that, upon execution by the one or more computer processors, implements any of the methods or techniques above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a block diagram of an environment for analyzing variants.

FIG. 2A shows an example of a flowchart illustrating a method for analyzing variants.

FIG. 2B shows an example of a flowchart illustrating another method for analyzing variants.

FIG. 3 shows an example of a computer system that is programmed or otherwise configured to implement methods provided herein.

FIG. 4 shows an example of a random forest with a plurality of decision trees.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

With the increasing volume of genetic data being generated, the burden on expert reviewers of genetic data has become substantial. For example, for genotype-first assays (e.g., exome or genome), there is an unmet need to algorithmically triage variants for “classical” analysis.

In some cases, the systems, the methods, the computer-readable media, and the techniques disclosed herein provide the Model for Inference of Mutation Impact (MIMI) Variant Ranker, which refers to algorithms that combine multiple features (input values to the machine learning model of the MIMI Variant Ranker; each model uses a list of features) related to the variants in a subject (e.g., patient) case and leverages machine learning (ML) to rank the relevance of variants within an exome-based or genome-based test. In some cases, the MIMI Variant Ranker may help to improve (e.g., optimize) the variant review process by automating the variant scoring process, thereby enabling genetic experts to focus on more complex cases and critical decision-making tasks. The MIMI Variant Ranker is designed to prioritize variants and encompasses the training and testing of the MIMI Variant Ranker machine learning model (a machine learning model configured to prioritize variants from exome and genome tests) and execution of the model via the MIMI command line interface.

Advantageously, the systems, the methods, the computer-readable media, and the techniques disclosed herein reliable predictions for prioritizing single nucleotide variant (SNV) and small insertion/deletion variants (INDELs) for exome- or genome-based testing. Further advantageously, the systems, the methods, the computer-readable media, and the techniques disclosed herein help to reduce (e.g., minimize) errors caused by missed diagnostic variants. Further advantageously, the systems, the methods, the computer-readable media, and the techniques disclosed herein significantly reduce the time and effort expended by clinical analysts manually reviewing a large set of variants.

In some cases, the systems, the methods, the computer-readable media, and the techniques disclosed herein may provide an interface (e.g., a command-line interface) for facilitating user interaction with the MIMI Variant Ranker. In some cases, the systems, the methods, the computer-readable media, and the techniques disclosed herein may provide machine learning techniques for implementing the MIMI Variant Ranker, including training datasets and feature collection in the GOR platform. Note that the GOR format is a file format for variant tables, including columns for chromosomes, positions, reference alleles, and variant alleles (e.g., ordered by chromosome and position). While disclosed herein with respect to the GOR platform, note that in some cases, the systems, the methods, the computer-readable media, and the techniques disclosed herein may implement any number of similar file formats for storing variants and related information. In some cases, the systems, the methods, the computer-readable media, and the techniques disclosed herein may provide real-world data demonstrating efficacy of the MIMI Variant Ranker (e.g., for 6,740 positive cases, as disclosed herein).

### Examples of Variant Ranking

In some cases, the systems, the methods, the computer-readable media, and the techniques disclosed herein may comprise providing the MIMI Variant Ranker. In some cases, the MIMI Variant Ranker infers the ranked relevance of a case variant in the context of a disease diagnosis. For example, the disease diagnosis may be for Mendelian diseases. Further, the disease diagnosis may be downstream of samples of whole genome sequencing (WGS) or whole exome sequencing (WES). In some cases, the ranking produced by the MIMI Variant Ranker may also be referred as variant prioritization.

In some cases, the top ranked variant or variants by the MIMI Variant Ranker may be displayed (e.g., presented or indicated). For example, the top ranked variant or variants may be displayed with a comparative sequence analysis (CSA) tool, such as, the Gregor CSA. While disclosed herein with respect to the Gregor CSA, in some cases, the systems, the methods, the computer-readable media, and the techniques disclosed herein may leverage any number of similar software platforms designed to handle sample onboarding, variant annotation, variant interpretation, and report writing. The MIMI Variant Ranker may use a multidimensional, wholistic view of many possible features to predict the relative relevance of each case variant using parameters learned during the training procedure. In some cases, the Gregor workflow template, Gregor-MIMI Variant Ranker (or the like), may be used generates all the features (inputs) except for Multiscore, which may be run separately. As disclosed herein, Multiscore is a software tool for combining multiple measure of phenotypic similarity scoring to predict overall relevance of variant data in an exome-based test or genome-based test. In some cases, the processes may be handled within the workflow-process-case Gregor Nextflow pipeline, a pipeline that executes the operations to analyze variants in a subject (e.g., patient) case.

In some cases, the top-k ranked variant results of the MIMI Ranker may be displayed to the clinical analyst within the CSA tool (e.g., Gregor CSA). In some cases, displaying the top-k ranked variant results includes displaying the variants that satisfy some threshold (e.g., the top N variants, variants satisfying a particular score, etc.). Through the MIMI Variant Ranker achieving a strong recall at a given k (“recall at k”) the time and effort expended by clinical analysts may be reduced via the MIMI Variant Ranker reducing the list of variants to manually review. Note, recall at k may be a performance metric for algorithmic ranking, defined as “the number of relevant items in top k divided by the total number of relevant items.”

### Model Overview

In some cases, the systems, the methods, the computer-readable media, and the techniques disclosed herein provide the MIMI Variant Ranker configured to prioritize variants likely to be relevant to the diagnosis of Mendelian disease within a specific case. In practice, the Gregor Nextflow pipeline workflow-process-case (or the like) may help to execute the MIMI Variant Ranker. For example, the MIMI Variant Ranker may be executed on a command-line interface (CLI). In this example of the MIMI Variant Ranker as a CLI software tool, an app within the machine learning model may (A) convert variants from GOR-format variant details to Avro schema, (B) prioritize variants in a case using the MIMI ML model, and (C) converts prioritized variants from Avro schema to GOR-format.

In some cases, the MIMI Variant Ranker may be executed by providing a table of case variants. In some cases, the case variants may be annotated with features (e.g., by the Gregor-MIMI Variant Ranker workflow, or the like). In some cases, the output of the MIMI Variant Ranker may be a scored list of case variants.

In some cases, the features for each variant may be organized into different categories. For example, the top-level category may be the distinction between “variant-level” and “case-level” features. Variant-level features may be features associated with the variant. These features may be relevant to the variant “in a vacuum;” in other words, the features may be true regardless of any information about the subject (e.g., patient) or the patient's family. Examples of variant-level features may include variant population frequencies and variant effect prediction. On the other hand, case-level features may connect the variant to the subject (e.g., patient) and patient's family. Examples of case-level features may include variant segregation data or inheritance data and the connection between a variant and the phenotypes associated with the subject (e.g., patient).

In some cases, past observations of case-level data may translate to variant-level data for a current case of interest, such as a “variant previously observed to have appropriate segregation within affected individuals in a family.” Further categorization of features are disclosed elsewhere herein.

In some cases, the MIMI Variant Ranker may leverage a model. For example, the model may be a machine learning model. The model may have learned to rank variants in a case via a truth set of training dataset. This learned information may be captured within a trained machine learning model file. The machine learning model for the MIMI Variant Ranker may be hosted, in some cases, hosted on a server or cloud storage.

In some cases, the MIMI Variant Ranker machine learning model may be retrained and tested over various weights and biases (W&B). Note that W&B data may be stored in a directory including data files stored in cloud storage and tracked by W&B. In the MIMI Variant Ranker, the W&B data may be trained by the machine learning model files of the MIMI Variant Ranker. In W&B, this may be referred to as an “artifact.”

In some cases, as the machine learning model is retrained, the machine learning model may be redeployed into production workflows to maintain the predictive performance of the MIMI Variant Ranker. This cadence for retraining and redeploying may be based on model drift, time elapsed, cases added, feature engineering, etc. In some cases, retrained machine learning model for the MIMI Variant Ranker may be redeployed per regression testing and model rollback contingencies.

In some cases, The machine learning model for the MIMI Variant Ranker may be optimized using hyperparameter sweeps and feature engineering experiments. For example, as used by the machine learning model, hyperparameters may be the parameters that control the learning process of the machine learning model. These hyperparameters may control, for example, the depth of decision trees used during training of the machine learning model. A hyperparameter sweep may be a set of model training and testing where the hyperparameters are tuned over set ranges. Automated hyperparameter sweeps may leverage defined optimization algorithms to efficiently generate and identify the machine learning models with improved (e.g., maximized) performance to use with the MIMI Variant Ranker. Note, a sweep may be a machine learning experiment comprising many runs of model training and testing, where the goal of the experiment is to deliver a machine learning model configured for (e.g., optimized for) analytical performance. During a sweep, many machine learning models are trained with adjusted hyperparameters and then tested for performance. As the sweep progresses, the hyperparameters are tuned to favor the values that deliver the strongest performance.

In some cases, feature engineering experiments to use with the MIMI Variant Ranker may include determining or monitoring the influence of features on machine learning model performance using machine learning interpretability algorithms. These algorithms may provide, for example, insights into the decision making of a machine learning model. For example, feature engineering may include determining an answer to the question of “does this particular machine learning model's performance improve when a feature is added to or removed from the data set?” Further details of training, validating, improving, etc. the machine learning model used for the MIMI Variant Ranker are disclosed elsewhere herein.

### Prioritization

In some cases, the MIMI Variant Ranker may use a machine learning model from the “Learning to Rank” family of algorithms. This indicates that the variant scores generated by the machine learning model may reflect a prediction of ranked relevancies. As in, the scores may be predictive of relevance with respect to other variants in the case; however, the score may not be meaningful independently. Notably, this is distinct from traditional classification or regression algorithms that make predictions of an outcome of a single data point. In other words, the MIMI Variant Ranker may generate predictions that may be interpreted as “this variant has the highest likelihood of being the causative mutation in this patient case” and may not be interpreted as “this variant has an X % likelihood of being the causative mutation in this patient case.” Accordingly, interpreting the likelihood that a prioritized variant causes disease leverage clinical knowledge and experience.

However, in other cases, the MIMI Variant Ranker may use a machine learning model configured to generate predictions that are meaningfully independent, such as “this variant has an X % likelihood of being the causative mutation in this patient case.”

### Technical Performance

In some cases, the MIMI Variant Ranker may rank large numbers of variants. For example, with single nucleotide variants (SNVs) and small indels, the MIMI Variant Ranker may be configured to rank 20,000 cases per quarter by the end of 2024, with a median of 4,800 variants ranked per case and the middle 80% of cases containing a total of 4200-9100 variants (reported to two significant figures). These performance metrics may be based on the growth targets for WES/WGS testing and the variant filtering from XoneAnalyzer (XA).

In some cases, MIMI Variant Ranker may be a script that may be pulled into the Gregor NextFlow pipeline, such as, through a CLI. This indicates that independent copies of MIMI Variant Ranker may be pulled into as many concurrent Gregor Nextflow instances as desired by users with little to no strain on the MIMI Variant Ranker. Notably, this is distinct from an application programming interface (API) that may act as a “service” that responds to many requests at once, from a centralized location.

In some cases, the MIMI Variant Ranker experiments demonstrate that the MIMI Variant Ranker can rank approximately 2,000 variants in 33 seconds on within the Gregor NextFlow environment. Additional details on technical performance, including case volume stress testing and execution run times, are disclosed elsewhere herein.

### Analytical Performance

In some cases, the MIMI Variant Ranker ranks the diagnostic variants into the top 3 in 90% of relevant exome-sequencing and genome sequencing tests. This may be based on a proof-of-concept (POC) study of 6,740 retrospective positive cases, which is further disclosed elsewhere herein.

### Examples of Technical Systems

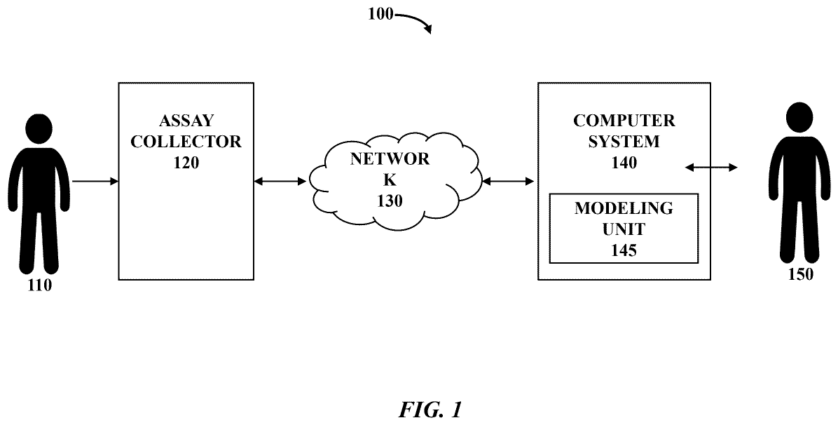

FIG. 1 is a block diagram of an environment 100 for analyzing variants for a subject 110. The environment 100 of FIG. 1 includes one or more data-collection instruments, illustrated an assay collector, 120. As illustrated, a network 130 connects the assay collector 120 and a computer system 140 having a modeling unit 145. The computer system 140 may be accessible to a human operator 150, who may be a physician, genetic researcher, etc.

In some cases, the assay collector 120 may comprise one or more devices, including recording equipment (e.g., medical or scientific equipment) to generate assay data that may then entered into an electronic device. In other cases the assay collector 120 may be an integrated device that is both configured to generate the assay data and then store and transmit the assay data. At a high level, the assay collector 120 may be used by one or more users (e.g., care providers, researchers, family members of a subject (e.g., patient), a subject (e.g., patient) themselves, etc.) to collect or provide the assay data about the subject 110 to the computer system 140.

The assay collector 120 may comprise one or more electronic devices. In some cases, the electronic devices may be desktop computers. In some cases, the electronic devices may be mobile devices. For example, the mobile devices may be smartphones, tablets, etc. In some cases, the electronic devices may be medical devices or research devices. In some cases, the electronic devices may be wearable or ambient devices or sensors. For example, the wearable or ambient devices or sensors may be smartwatches, smartbands (e.g., a Fitbit® device), smartclothing, smart jewelry, smartshoes, environmental sensors, or the like. In some cases, the electronic devices may be a laptop or desktop computer. In general, the assay collector 120 may be any device suitable for collecting assay data, health data (e.g., wearable data, ambient device datal sensor data, responses to queries, geographical data, demographical data, medical data, etc.) For example, the assay collector 120 may comprise any one or more of desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, vehicles, televisions, exercise equipment, video players, digital music players, booklet tablet computers, slate tablet computers, convertible tablet computers, or the like.

As described, the assay collector 120 comprise data-collection instruments that can be medical, research, wearable, or ambient devices or sensors or other devices capable of providing health data about one or more users, including assay data. For example, the assay collector 120 may comprise, in addition to a device configured to collect the assay data, any number of medical or health devices such as a fitness tracker, a pedometer, a wrist-worn sleep tracker, a sleep-tracking mattress pad, a smart scale, a blood pressure monitor, an ambient air particle sensor, a CPAP machine, a smart watch, smartphone, or mobile device (e.g., a tablet computer or a personal digital assistant (PDA)) with physical statistic monitoring functionality. In some cases, the subject 110 may be associated with multiple devices comprising the assay collector 120, measuring overlapping or distinct physical statistics about the subject 110. The assay data 120 gathered by the assay collector 120 can be sent to the computer system 140 directly from the assay collector 120, manually uploaded to the computer system 140 (e.g., by the subject 110, a care provider, a family member, a researcher, etc.), or transmitted via a third-party system to the computer system 140. 130. In some cases, a user (e.g., the subject 110, a care provider, a family member, a researcher, the human operator 150, etc.) can interact with the computer system 140 via the assay collector 120, or vice-versa. For example, the user may be able to update information about the subject (e.g., patient) 110 (e.g., the assay data, physical statistics, health conditions, behavior, etc.) stored in the computer system 140 via the assay collector 120. In some cases, the user can interact with the assay collector 120 via an external device (not shown; e.g., a laptop, a smartphone, etc.). For example, the user may configure settings of the assay collector 120 through the external device (e.g., turn one or more devices of the assay collector 120 on/off, change a sampling rate, etc.). In some cases, the user may further be able to provide feedback relating to one or more estimates or predictions generated using the computer system 140 or manually report health information to the computer system 140 via the assay collector 120. For example, in some cases, the user may, e.g., through one or more devices of the assay collector 120, report to the computer system 140 that the subject 110 has received a treatment or intervention, has a certain health condition, has certain demographics/medical history, etc.

In some cases, the assay collector 120 may be a separate device (or separate devices) from the computer system 140. In such cases, the assay collector 120 may transmit the assay data to the computer system 140. Transmission may be accomplished via the network 130. Alternatively or in addition, transmission may be accomplished via a wired connection or a local connection (e.g., Bluetooth). In other cases, the assay collector 120 may be at least partially integrated into the computer system 140, as a common device. In these cases, the network 130 may not be needed to share the assay data between the assay collector 120 and the computer system 140.

As illustrated, the assay collector 120 (and, in some cases, users of the assay collector 120) may communicate with the computer system 140 over the network 130. The network 130 may be a network or system of networks connecting the computer system 140 to the assay collector 120. The network 130 may comprise any combination of local area or wide area networks, using wired or wireless communication systems. In some cases, the network 130 uses standard communications technologies or protocols. For example, the network 130 can include communication links using technologies such as Ethernet, 3G, 4G, 5G, CDMA, WIFI, Bluetooth, etc. Data or information exchanged over the network 130 may be represented using any suitable format, such as hypertext markup language (HTML) or extensible markup language (XML). In some cases, all or some of the communication links of the network 130 may be encrypted using any suitable technique or techniques. In some cases, the network 130 also facilitates communication between the computer system 140, the assay collector 120, and other entities of the environment 100 such as the modeling unit 145, users (not shown) or other external devices (not shown).

The computer system 140 may comprise computer devices such as a server, server cluster, distributed server, or cloud-based server capable of predicting health conditions, statistics, risks, etc. For example, the computer system 140, using the modeling unit 145, may perform the MIMI Variant Ranker. In some cases, the modeling unit 145 may select a subset or variants or rank a subset of variants of a total batch of variants containing, e.g., thousands of variants in the assay data. In some cases, performing the MIMI Variant Ranker for the subject 110 may inform the generation of the personalized treatment or intervention recommendations for the subject 110.

The computer system 140 can analyze received the assay data to extract learned features or generate a learned representation of the assay data using the modeling unit 145. In some cases, the learned representation generated by the modeling unit 145 may store a transformed, modified, or compressed version of the assay data. This version of the assay data (or wearable or ambient device or sensor data) may preserve richness of information and useful features that may be used to identify trends or outliers among the assay data, predict variant relevance, segment, cluster, or categorize variants, etc.

In some cases, outputs (e.g., predictions of variant rankings) from the computer system 140 may be shared. For example, the outputs from the computer system 140 may be shared with the corresponding user, such as the subject 110 or the human operator 150. The outputs from the computer system 140 may be shared with the human operator 150, who may comprise researchers or a laboratory (e.g., for studying health conditions), etc. with consent from the subject 110. In another example, the outputs from the computer system 140 may be shared with the human operator 150, who may comprise healthcare providers (e.g., the primary care physician, nurse, caregiver, etc. of the subject 110), with consent from the subject 110. In cases where the outputs from the computer system 140 are shared with the healthcare providers, the healthcare providers may maintain a computing system such as a server, set of servers, server cluster, etc. which can create or modify an individual treatment plan or perform interventions based on output using the computer system 140. For example, the computing system of the healthcare providers can be managed by a medical provider, doctor, or other entity providing medical care to the subject 140.

### Formats and Schemas

In some cases, the assay data, or data associated thereof, may be comprise information corresponding to chromosomes, variant position, reference allele, called allele, etc. For example, the assay data, or the data associated thereof, may be stored in a GOR format, e.g., in a table-separated values (TSV) file type. In some cases, each column of the TSV file may correspond to a different parameter (e.g., chromosome, variant position, reference allele, called allele, etc.). In some cases, the GOR format may implement chromosome and position in ascending order.

In some cases, the MIMI Variant Ranker uses Avro (https://avro.apache.org/), a Python library that facilitates fast, lightweight, and controlled input/output (I/O) by combining file compression (serialization) with data contracts. Although disclosed herein with respect to Avro, in some cases, the systems, the methods, the computer-readable media, and the techniques disclosed herein may use a similar tool for facilitating file serialization (compression) and data contracts via a schema for file input and output (I/O).

Avro may use schema based on Pydantic models to establish data contracts between systems. The contract may be validated at each level of the schema, and any deviation from the expected schema may raise a ValidationError. Avro schema may be defined in *.avsc files, which are json files following Avro format. In MIMI Variant Ranker, the input and output schema may be respectively defined by: app/mimi_ranker/tests/cli/score/schema/test_data/input.avsc; and app/mimi_ranker/tests/cli/score/schema/test_data/output.avsc. In some cases, the .avsc files may be paired with Avro Pydantic model objects dataclasses_avroschema.pydantic.AvroBaseModel from the Python library dataclasses_avroschema (https://pypi.org/project/dataclasses-avroschema/). The serialized files may be in the Avro format with *.avro file extensions.

In some cases, the MIMI Variant Ranker has helper functions to convert files between the Pydantic model classes and avsc j son files. This may allow for a prescribed procedure to make updates to the schema, for example, as features are added or removed from the machine learning model of the MIMI Variant Ranker. For instructions on updating the schema, see the SCHEMA README of (https://github.com/GeneDx/mlplatform/blob/main/app/mimi_ranker/src/mimi_ranker/cli/score/schema/README.md).

In some cases, the case input may be defined as:
A list of case records, each defined by:
mimi_ranker.cli.score.schema.input.CaseInputRecord. This record contains two fields:
   i. case identifier pn
   ii. a list of variant records each, defined by mimi_ranker.cli.score.schema.input.VariantRecordInput. This sub-record of CaseInputRecord contains two fields:
      1. the definition of the variant mimi_ranker.cli.score.schema.common.VariantDefinition containing chromosome, position, reference allele, called allele, gene symbol, and hgnc id.
      2. the features of the variant mimi_ranker.cli.score.schema.input.VariantFeatures containing the features the MIMI ML model uses to infer the priority of the variants in the case.

In some cases, the case output may be defined as:
A list of case records, each defined by:
mimi_ranker.cli.score.schema.output.CaseOutputRecord. This record contains two fields:
   i. case identifier pn.
   ii. a list of variant records, each defined by mimi_ranker.cli.score.schema.output.VariantRecordOutput. This sub-record of CaseOutputRecord contains two fields:
      1. the same definition of the variant VariantDefinition shown above.
      2. the variant result mimi_ranker.cli.score.schema.output.VariantResults containing the score plus metadata.

Note that the input and the output schema may each be in a list of CaseInputRecord and CaseOutputRecord respectively, therefore, the MIMI Variant Ranker may analyze a single case or analyze multiple cases individually with a single three step call detailed in the subsequent section.

### Operation and Input-Output

In some cases, the MIMI Variant Ranker is designed to prioritize SNVs and small INDELs called by GATK HapotypeCaller in WES/WGS cases. In some cases, multi-proband cases may be excluded from the training, testing, and holdout sets and may be excluded from analysis with the MIMI Variant Ranker. Note that currently, Multiscore may also exclude multi-proband cases.

In some cases, the MIMI Variant Ranker may be called within the Gregor NEXTFLOW pipeline. In some cases, variant filtering and annotation may be handled by the GREGOR-MIMI Gregor workflow template. The GREGOR-MIMI workflow template (a template that builds all the feature data expected by the MIMI Variant Ranker CLI) is maintained in the GeneDx GitLab cla-queries repository at: https://gitlab.com/wuxi-nextcode/cla/cla-queries/-/blob/master/templates/gdx/gregor_mimi.ftl.yml. Future updates to the MIMI Variant Ranker machine learning model may leverage coordinated updates to Gregor-MIMI workflow template.

In some cases, the Gregor Nextflow pipeline process-case-workflow may pass the filtered and annotated case variants to the MIMI Variant Ranker for formatting and prioritization. The MIMI Variant Ranker may perform the following three operations: (A) GOR to input Avro transformation; (B) variant scoring by the MIMI Variant Ranker; and (C) output Avro schema to GOR transformation. These commands may be handled using the typer python library (https://typer.tiangolo.com/) converting the methods to typer. Typer objects as Application Programming Interface (API) calls. Example commands to execute these three steps are provided in the MIMI Ranker README (https://github.com/GeneDx/mlplatform/blob/main/app/mimi_ranker/README.md). Additional details regarding the three operations of the MIMI Variant Ranker are provided below.

GOR to input Avro transformation: This operation is executed via the method mimi_ranker.cli.score.schema.schema_helpers. convert_from_tsv_to_input_schema( ) In this operation, the case variant file in GOR format is transformed into a list of case records in the Avro schema CaseInputRecord. The operations are:
1. Read the GOR case variant file.
2. Group the variants by case identifier pn, and for each case:
   a. Convert from GOR format pandas dataframe to json format.
   b. Convert from json format to the input schema; a list of CaseInputRecord records. Note that the script validates the contract and raises a ValidationError if the input GOR file does not contain the correct columns defined in CaseInputRecord.
3. Write the input case variant data to a serialized avro file.

Variant scoring by the MIMI Variant Ranker: This step is executed via the method mimi_ranker.cli.score.cli.cases( ) In this operation, the variants from each case are ranked via inference by the MIMI ML model. The operations may include:
1. Load the MIMI ML model. The production model can either be downloaded from W&B data remote storage or loaded from local storage.
2. Read the input avro file. Each case is defined by a single CaseInputRecord record.
3. For each case, score variants with the MIMI ML model using the features defined in VariantFeatures and ranked via the method mimi_ranker.cli.score.mimi utils.inference_pipeline( )
4. Write the scored case variant data (a list of CaseOutputRecord records) to a serialized avro file.

Output Avro schema to GOR transformation: This operation is executed via the method mimi_ranker.cli.score.schema.build_output. convert_from_output_schema_to_tsv( ). In this operation, the scored case variant file in Avro format is transformed back into GOR format. The operations may include:
1. Read the serialized output scored case variants avro file.
2. For each case, convert from output avro format (a list of CaseInputRecord records) to json format to pandas dataframe (https://pandas.pydata.org/) table format.
3. Write the pandas dataframe containing output scored case variant data to GOR format.

In some cases, the holdout set may be used for model validation. For example, retrospective cases may be used to evaluate the MIMI Variant Ranker. Disease Phenotype Resource (DiPR) case table cases with at least one positive SNV or INDEL may be used. The holdout set may supplement the testing dataset and establish a set of cases from a time window that is distinct from the training dataset. If the model performance is observed to be correlated with the date the case was closed, it suggests that (A) there is “model drift” because the most recent data is different from the training dataset, or (B) there may be a “data bleed” between training and testing datasets. This also mimics a production future state, where the live cases are from a point in time that comes later than the training dataset. Note, DiPR may be a data table of generated probands in Exome Sequenced cases. The DiPR case table may be updated periodically (e.g., monthly at end of month based on date of case closed). The cases are in DiPR may be excluded if any of the following are true: healthy probands, prenatal probands, multiple report cases, or consent for R&D was declined.

### Features

In some cases, the systems, the methods, the computer-readable media, and the techniques disclosed herein may implement machine learning features. In machine learning, features may be the observables that are used as inputs for training, testing, and usage of a model. In some cases, the machine learning model of the MIMI Variant Ranker may be extensible and some features may be added, modified, or subtracted, as needed. In some cases, the avro schema VariantFeatures may define the feature list. New or modified features may use coordinated updates to the Gregor-MIMI workflow template. However, in some cases, features may be subtracted without such coordination by setting a default feature value to null in the VariantFeatures definition and updating the inference_pipeline( ) method to handle the change. Note that the null default value may be used because any extra fields may violate the contract (e.g., in the same fashion that missing fields may violate the contract, see Formats and Schemas section above). Removing the feature from the schema completely may require updates to the Gregor-MIMI workflow template.

In some cases, updating the feature list may include updating or retraining the MIMI Variant Ranker machine learning model. And, as with the retraining of the MIMI Variant Ranker machine learning model in hyperparameter sweeps (see below), deployment of a new machine learning model into production may trigger a minor release (or rerelease) of the MIMI Variant Ranker. In this case, a regression test on the prioritization of positive variants in retrospective cases may be performed to ensure the performance of the model is maintained or improved. The version of the machine learning model of the MIMI Variant Ranker and the version of the MIMI Variant Ranker itself may be output by a tool, and may use the GREGOR environment to perform a saving function.

In some cases, the MIMI Variant Ranker may comprise many features (e.g., at least about: 10, 20, 50, 100, 250, 500, 1000, 5000, 10000, etc. features). For example, in the below feature mapping, the MIMI Variant Ranker machine learning model may comprise about 114 features, divided into “variant-level” and “case-level” categories. The variety of sources and methods for feature generation may use the below further categorization:
1. Variant-level
   a. Population frequency
      i. Unaffected populations
         1. GnomAD v2.1.1 (https://gnomad.broadinstitute.org/)
         2. GeneDx unaffected patient population
         3. GnomAD-GeneDx combination
      ii. Affected populations
         1. GeneDx affected patient population
   b. VEP-determined variant consequence (https://useast.ensembl.org/info/docs/tools/vep/index.html)
   c. Known variant classification
      i. GeneDx classification
      ii. ClinVar classification (two stars or more) (https://www.ncbi.nlm.nih.gov/clinvar/docs/review_status/)
   d. (Gene-level) Broad Institute gene constraint scores (https://gnomad.broadinstitute.org/help/constraint)
   e. (Local-level) variant lies in RepeatMasker “repeat region” (https://www.repeatmasker.org/)
   f. In silico scores
      i. SpliceAI (https://github.com/Illumina/SpliceAI)
      ii. REVEL (https://sites.google.com/site/revelgenomics/)
      iii. Provean (https://www.jcvi.org/research/provean)
2. Case-level
   a. Inheritance pattern of variant(s)
   b. Phenotype matching
      i. Gene-level Fisher-exact GWAS associations
      ii. Variant-level Fisher-exact GWAS associations
      iii. Multiscore scores
      iv. PhRank scores (https://github.com/Mizari/phrank)
3. Variant-level or case-level
   a. InterVar ACMG logic (https://wintervar.wglab.org/)

In some cases, the systems, the methods, the computer-readable media, and the techniques disclosed herein may implement a GORPipe query. For example, the logic for these features may be executed with a GORPipe query to annotate the case variants for the cases, defining the training dataset and testing dataset. The case variants are the variants stored in XA and uploaded to GORdb. The annotations come from the production reference files stored in GORdb, with a few exceptions. First, the Multiscore values may be generated in monthly batches, uploaded to GOR, and optimized for incorporation into the query (note that in practice, the Multiscore values may be generated live via the Multiscore CLI). Second, positive/non-positive variant labels may be generated from the Disease Phenotype Resource (DiPR) case table. The positive variants were normalized and joined to the GORdb variants (note that in practice, the labels may not be known during live case analysis, because the outcome of the case has not been determined). The labels may be the value the machine learning model of the MIMI Variant Ranker is designed to predict. In this case, the labels are defined by a “truth set” of diagnostic variants and non-diagnostic variants from internal positive cases.

### Training Dataset and Testing Dataset

In some cases, the training dataset in the MIMI Variant Ranker may include the cases and variants that the machine learning model of the MIMI Variant Ranker uses to learn to prioritize variants causing Mendelian disease in the subject (e.g., patient) cases. The outcome of applying the training dataset to the machine learning model's algorithm is the trained MIMI Variant Ranker. The testing dataset may include the cases and variants that are used to evaluate the performance of the MIMI Variant Ranker. Note, both the training and testing datasets may include positive cases from the DiPR case table.

In some cases, the testing dataset may include retrospective cases used to evaluate the machine learning model of the MIMI Variant Ranker with DiPR cases having at least one positive SNV or INDEL closed between. Note, part of the testing dataset may be randomly split from the same time window as the training dataset, and part is a holdout set from a distinct time window. In some cases, the training dataset may include retrospective cases used to train the machine learning model of the MIMI Variant Ranker with DiPR cases with at least one positive SNV or INDEL.

In some cases, the records in each case may be the case variants (SNVs and insertion/deletion variant (INDEL)) that were uploaded to XomeAnalyzer (XA). Future the machine learning model training for the MIMI Variant Ranker may include case variants from samples that have been onboarded into GORdb directly. This may use either (A) onboarding of sufficient legacy sample data, or (B) onboarding of sufficient live sample data.

In some cases, the labels in the training and testing datasets are the historical positive/non-positive (1/0) relevance values of variants per the clinical diagnostic workflows. For example, a positive variant may be assigned a relevance of “1—Causative Mutation” and referred to as a “cat1” variant. Only positive cases were used in training and testing since positive variants may be found in positive cases. Note, other relevance values may be investigated in the UAT, such as “2—Possibly Associated” and “3—Candidate Gene.” Positive cases may be cases where one or more variants is assigned a relevance of “Causative Mutation” (a.k.a., “cat1”), as in, a case where a molecular diagnosis was made. Positive variants may be variants that are assigned a relevance of “1—Causative Mutation” (a.k.a., “cat1”) for a particular case.

As reflected in Table 1, positive DiPR cases closed between Nov. 1, 2021-7/31/2023 were collected. This case set includes a mix of trios, duos, and singletons (multiple proband cases were not included). Cases in this time range were randomly assigned to the training or testing datasets in a 75% to 25% ratio. The testing dataset was supplemented by “true hold-out set” of cases closed between Aug. 1, 2023-4/30/2024. Only cases with one or more positive SNVs or INDELs were retained. The training and testing dataset sets are summarized in Table 1. As shown in Table 1, Single diagnosis, Category 1 (SDC1) represents single diagnosis cat1 case (a single gene was assigned a positive finding) and Multiple diagnoses, Category 1 (MDC1) represents multiple diagnosis cat1 cases (multiple genes were assigned a positive finding). In other words, MDC1 represents a subset of positive cases where variants from multiple genes have been assigned a relevance of “1—Causative Mutation” and SDC1 represents a subset of positive cases where one or more variants from a single gene have been assigned a relevance of “1—Causative Mutation.”

TABLE 1
The MIMI Variant Ranker case training and testing dataset set
summary. The date range of the training and testing datasets
overlap. The date range of the holdout set is distinct from
the training and testing datasets. The testing dataset is
supplemented by the holdout set during model testing.

| Data Set | Case Closed Date | SDC1/MDC | Number of |
|---|---|---|---|
| Training | Nov. 1, 2021- | SDC1 | 7,299 |
| Training | Nov. 1, 2021- | MDC1 | 181 |
| Testing | Nov. 1, 2021- | SDC1 | 2,444 |
| Testing | Nov. 1, 2021- | MDC1 | 49 |
| Holdout | Aug. 1, 2023-Jan. 31, 2024 | SDC1 | 4,132 |
| Holdout | Aug. 1, 2023-Jan. 31, 2024 | MDC1 | 107 |

### Machine Learning Model for MIMI Variant Ranker

In some cases, the machine learning model for the MIMI Variant Ranker may be the algorithm trained and used to predict the correct prioritization of SNVs and INDELs in a WES/WGS case. To maximize performance and prevent “model drift” (gradual machine learning model performance loss over time), the machine learning model for the MIMI Variant Ranker may be continually retrained and reevaluated using hyperparameter sweeps and tested on retrospective case data. In some cases, after regression testing, a machine learning model (e.g., the optimal model) may be selected and tagged as the “production” model in W&B. The selected machine learning model for MIMI Variant Ranker may be used in production. The production model may be downloaded into the cloud-hosted storage of the Gregor Nextflow pipeline via tooling in the mlplatform code repository. In some cases, promoting a new machine learning model may trigger a minor release (or rerelease) of the MIMI Variant Ranker.

In some cases, the machine learning model of the MIMI Variant Ranker may be the LAMDAMART (https://dl.acm.org/doi/10.5555/2976456.2976481) ranking model implemented in the XGBoost python package as XGBoost. XGBRanker (https://xgboost.readthedocs.io/en/stable/python/python_api.html). The objective function for the machine learning model during training may be Normalized Discounted Cumulative Gain (NDCG). NDCG represents a performance metric used in algorithmic ranking, where NDCG is maximized when the algorithmically predicted rank of records (e.g., variants) corresponds with the truth-set relevance of the records. As disclosed herein, NDCG may be the objective function used during machine learning model training. In some cases, the trained machine learning model file may be stored as a json. Future versions of the MIMI Variant Ranker may leverage other model types. Note, the XGBoost. XGBRanker may be compared against other ranking models during training sweeps.

### Failure Modes and Mitigations

In some cases, software dependency version control within the mlplatform repository may be handled using poetry (https://python-poetry.org/) and the project-level versions are stored as: (A) MIMI Ranker: app/mimi_ranker/pyproject.toml, and (B) MIMI Trainer: lib/mimi_trainer/pyproject.toml. In some cases, a GitHub repository for each package may be found at (A) MIMI Ranker: https://github.com/GeneDx/mlplatform/app/mimi_ranker, and (B) MIMI Trainer: https://github.com/GeneDx/mlplatform/app/mimi trainer

In some cases, testing may be performed using the pytest testing framework (www.pytest.org), with current test coverage of about 92% (test coverage may refer to the percentage of the lines of code that are executed when the testing framework is run).

In some cases, the MIMI Variant Ranker may be stored in the ML Platform Oracle Cloud Container Registry. Pointing Gregor to a new container image may facilitate a software rollback. In some cases, the machine learning model of the MIMI Variant Ranker may be managed in W&B. Pointing Gregor to a new W&B data version may facilitate a model rollback. In some cases, a compatibility map may be provided between GREGOR-MIMI workflow template, the MIMI Variant Ranker CLI, and the MIMI Variant Ranker machine learning model.

### Examples of Methods

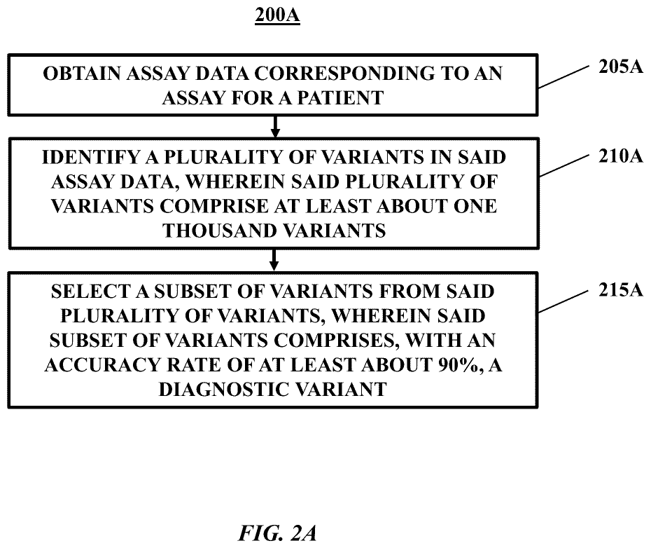

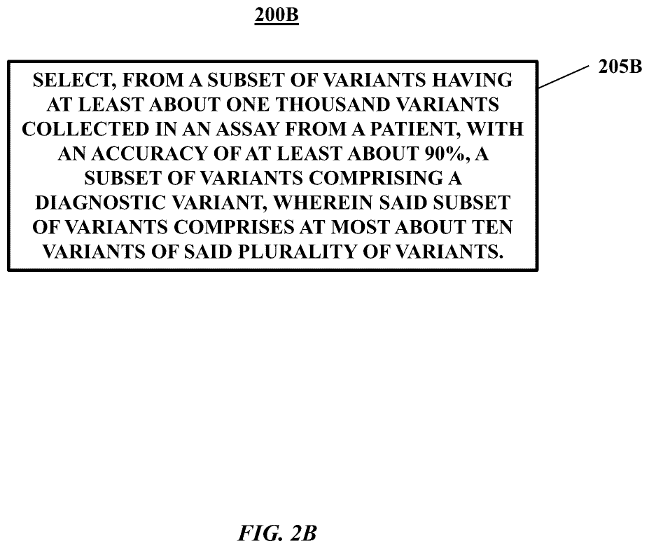

FIG. 2A shows an example of a flowchart illustrating a method 200A for analyzing variants, comprising: (A) obtaining assay data corresponding to an assay for a subject (e.g., patient) (block 205A); (B) identifying a plurality of variants in the assay data, wherein the plurality of variants comprise at least about one thousand variants (block 210A); and (C) selecting a subset of variants from the plurality of variants, wherein the subset of variants comprises, with an accuracy rate of at least about 90%, a diagnostic variant (block 215A). The method 200A may be implemented using one or more systems (e.g., hardware or software) described herein (e.g., the environment 100). The method 200A may implement one or more techniques or operations described herein.

In some cases, the block 205A comprises obtaining assay data corresponding to an assay for a subject (e.g., patient). In some cases, the assay comprises one or both of an exome-based test or a genome-based test. In some cases, the subject (e.g., patient) is suspected of or is diagnosed with a Mendelian disease.

In some cases, the block 210A comprises identifying a plurality of variants in the assay data, wherein the plurality of variants comprise at least about one thousand variants. In some cases, the plurality of variants comprise at least about three thousand variants. In some cases, the plurality of variants comprise at least about ten thousand variants.

In some cases, the block 215A comprises selecting a subset of variants from the plurality of variants, wherein the subset of variants comprises, with an accuracy rate of at least about 90%, a diagnostic variant. In some cases, selecting the subset of variants at the block 215A comprises generating, using a machine learning model, a score for each variant of the plurality of variants, wherein the score for each variant of the plurality of corresponds to a relevance of the variant to diagnosing the subject (e.g., patient). In some cases, selecting the subset of variants at the block 215A further comprises ranking the plurality of variants in a ranked order based at least in part on the score for each variant of the plurality of variants. In some cases, selecting the subset of variants at the block 215A uses a machine learning model that comprises one or more decision trees. In some cases, the machine learning model comprise a random forest. In some cases, the machine learning model is trained. In some cases, the subset of variants comprises at most about one hundred variants of the plurality of variants and the accuracy rate is at least about 99.9%. In some cases, the subset of variants comprises at most about twenty variants of the plurality of variants and the accuracy rate is at least about 99.9%. In some cases, the subset of variants comprises at most about ten variants of the plurality of variants and the accuracy rate is at least about 99.7%. In some cases, the subset of variants comprises at most about five variants of the plurality of variants and the accuracy rate is at least about 99%. In some cases, the subset of variants comprises at most about three variants of the plurality of variants and the accuracy rate is at least about 98%. In some cases, the diagnostic variant is a variant most relevant to diagnosing the subject (e.g., patient).

In some cases, the method 200A further comprises: causing a display to present the subset of variants to a user. In some cases, the user is one or both of a healthcare provider of the subject (e.g., patient) or a geneticist.

FIG. 2B shows an example of a flowchart illustrating a method 200B for analyzing a plurality of variants, having at least about one thousand variants, collected in an assay from a subject (e.g., patient), comprising: (A) selecting from the plurality of variants, with an accuracy of at least about 90%, a subset of variants comprising a diagnostic variant, wherein the subset of variants comprises at most about ten variants of the plurality of variants (block 205B). The method 200B may be implemented using one or more systems (e.g., hardware or software) described herein (e.g., the environment 100). The method 200B may implement one or more techniques or operations described herein.

In some cases, the block 205B comprises selecting from the plurality of variants, with an accuracy of at least about 90%, a subset of variants comprising a diagnostic variant, wherein the subset of variants comprises at most about ten variants of the plurality of variants. In some cases, selecting the subset of variants comprises: generating, using a machine learning model, a score for each variant of the plurality of variants, wherein the score for each variant of the plurality of corresponds to a relevance of the variant to diagnosing the subject (e.g., patient). In some cases, selecting the subset of variants further comprises: ranking the plurality of variants in a ranked order based at least in part on the score for each variant of the plurality of variants. In some cases, the plurality of variants comprise at least about three thousand variants. In some cases, the plurality of variants comprise at least about ten thousand variants. In some cases, selecting the subset of variants uses a machine learning model that comprises one or more decision trees. In some cases, the machine learning model comprise a random forest. In some cases, the machine learning model is trained. In some cases, the assay comprises one or both of an exome-based test or a genome-based test. In some cases, the subset of variants comprises at most about one hundred variants of the plurality of variants and the accuracy rate is at least about 99.9%. In some cases, the subset of variants comprises at most about twenty variants of the plurality of variants and the accuracy rate is at least about 99.9%. In some cases, the subset of variants comprises at most about ten variants of the plurality of variants and the accuracy rate is at least about 99.7%. In some cases, the subset of variants comprises at most about five variants of the plurality of variants and the accuracy rate is at least about 99%. In some cases, the subset of variants comprises at most about three variants of the plurality of variants and the accuracy rate is at least about 98%. In some cases, the subject (e.g., patient) is suspected of or is diagnosed with a Mendelian disease. In some cases, the diagnostic variant is a variant most relevant to diagnosing the subject (e.g., patient).

In some cases, the method 200B further comprises: causing a display to present the subset of variants to a user. In some cases, the user is one or both of a healthcare provider of the subject (e.g., patient) or a geneticist.

In some cases, any number of operations of the methods 200A or 200B may be added or removed. Further, the operations of the methods 200A or 200B may be performed in any order. Further, at least one of the operations of the methods 200A or 200B may be repeated, e.g., iteratively.

### Examples of Machine Learning Techniques

As disclosed throughout, in some cases, the systems, the methods, the computer-readable media, and the techniques disclosed herein may implement one or more machine learning techniques. In some cases, ML may generally involve identifying and recognizing patterns in existing data in order to facilitate making predictions for subsequent data. ML may include a ML model (which may include, for example, a ML algorithm). Machine learning, whether analytical or statistical in nature, may provide deductive or abductive inference based on real or simulated data. The ML model may be a trained model. ML techniques may comprise one or more supervised, semi-supervised, self-supervised, or unsupervised ML techniques. For example, an ML model (e.g., the machine learning model of the MIMI Variant Ranker) may be a trained model that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, residual neural networks, physics-informed neural networks, long short-term memory, deep belief networks, deep Boltzmann machines, deep convolutional neural networks, deep recurrent neural networks, large language models, transformer models, vision transformers, or generative adversarial networks.

In some cases, training the MIMI Variant Ranker machine learning model may include selecting one or more untrained data models to train using a training dataset set. The selected untrained data models may include any type of untrained ML models for supervised, semi-supervised, self-supervised, or unsupervised machine learning. The selected untrained data models may be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the ML model from the selected untrained data models may likewise be selected, such as limits on the ML model complexity or limits on the ML model refinement past a certain point. The ML model may be trained (e.g., via a computer system such as a server) using the training dataset set. In some cases, a first subset of the training dataset set may be selected to train the ML model. The selected untrained data models may then be trained on the first subset of training dataset set using appropriate ML techniques, based upon the type of ML model selected and any conditions specified for training the ML model. In some cases, due to the processing power requirements of training the ML model, the selected untrained data models may be trained using additional computing resources (e.g., cloud computing resources). Such training may continue, in some cases, until at least one aspect of the ML model is validated and meets selection criteria to be used as a predictive model.

In some cases, one or more aspects of the MIMI Variant Ranker machine learning model may be validated using a second subset of the training dataset set (e.g., distinct from the first subset of the training dataset set) to determine accuracy and robustness of the ML model. Such validation may include applying the ML model to the second subset of the training dataset set to make predictions derived from the second subset of the training dataset. The ML model may then be evaluated to determine whether performance is sufficient based upon the derived predictions. The sufficiency criteria applied to the ML model may vary depending upon the size of the training dataset set available for training, the performance of previous iterations of trained models, or user-specified performance requirements. If the ML model does not achieve sufficient performance, additional training may be performed. Additional training may include refinement of the ML model or retraining on a different first subset of the training dataset, after which the new ML model may again be validated and assessed. When the ML model has achieved sufficient performance, in some cases, the ML may be stored for present or future use. The ML model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of ML models may be stored for generating predictions under different sets of input data conditions. In some embodiments, the ML model may be stored in a database (e.g., associated with a server).

### Examples of Decision Trees and Random Forests

As described above, the machine learning model of the MIMI Variant Ranker may implement a decision tree. A decision tree may be a supervised ML algorithm that can be applied to both regression and classification problems. For example, a decision tree may grow from a root (base condition), and when it meets a condition (internal node/feature), it may split into multiple branches. The end of the branch that does not split anymore may be an outcome (leaf). A decision tree can be generated using a training dataset set according to the following operations: (A) starting from a root node (the entire dataset), the algorithm may split the dataset in two branches using a decision rule or branching criterion; (B) each of these two branches may generate a new child node; (C) for each new child node, the branching process may be repeated until the dataset cannot be split any further; (D) each branching criterion may be chosen to maximize information gain (e.g., a quantification of how much a branching criterion reduces a quantification of how mixed the labels are in the children nodes). The labels may be the data or the classification that is predicted by the decision tree.

A random forest regression is an extension of the decision tree model that tends to yield more robust predictions by stretching the use of the training dataset partition. Whereas a decision tree may make a single pass through the data, a random forest regression may bootstrap 50% of the data (e.g., with replacement) and build many trees. Rather than using all explanatory variables as candidates for splitting, a random subset of candidate variables may be used for splitting, which may enable trees that have different data and different variables (hence the term random). The predictions from the trees, which may be collectively referred to as the “forest,” may then be averaged to produce a final prediction. Many trees (e.g., ten trees, fifty trees, one hundred trees, one thousand trees, etc.) may be included in a random forest model, with a number (e.g., 3, 6, 10, etc.) of terms sampled per split, a minimum of number (e.g., 1, 2, 4, 10, etc.) of splits per tree, and a minimum split size (e.g., 16, 32, 64, 128, 256, etc.). Random forests may be trained in a similar way as decision trees. Specifically, training a random forest may include the following operations: (A) randomly select k features from the total number of features; (B) create a decision tree from these k features using the same operations as for generating a decision tree; and (C) repeat the previous two operations until a target number of trees is created.

As disclosed, a random forest classifier, which may comprise a plurality of decision trees where the output prediction may be the mode of the predicted classifications of the individual trees, can be helpful in reducing overfitting to training dataset. In some cases, an ensemble of decision trees can be constructed using a random subset of features at each split or decision node. The Gini criterion may be employed, in some cases, to choose the best partition, where decision nodes having the lowest calculated Gini impurity index are selected. The Gini impurity can be used, in some cases, as a criterion to find informative features based on which the splits in each decision tree may be constructed.

In some cases, each decision tree of a random forest may comprise one or more decision nodes, where each decision node specifies a predicate condition. For example, decision node may predicate the condition that, for a given dataset, the outcome to an question is a specific outcome. At each decision node, a decision tree can be split based on whether the predicate condition attached to the decision node holds true, leading to various prediction nodes. Each prediction node can comprise output values that represent “votes” for one or more of the classifications or conditions being evaluated by the assessment model. At prediction time, a “vote” can be taken over all of the decision trees, and the majority vote (or mode of the predicted classifications) can be output as the predicted classification.

In some cases, when the dataset being queried in the assessment model reaches a “leaf”, or a final prediction node with no further downstream splits, the output values of the leaf can be output as the votes for the particular decision tree. Since a random forest model comprises a plurality of decision trees, the final votes across all trees in the forest can be summed to yield the final votes and the corresponding classification of the subject. A large number of decision trees can help reduce overfitting of the assessment model to the training dataset, by reducing the variance of each individual decision tree. For example, an assessment model can comprise, for example, at least about 3 decision trees, at least about 5 decision trees, at least about 10 decision trees, at least about 20 decision trees, at least about 50 decision trees, at least about 100 decision trees, etc.

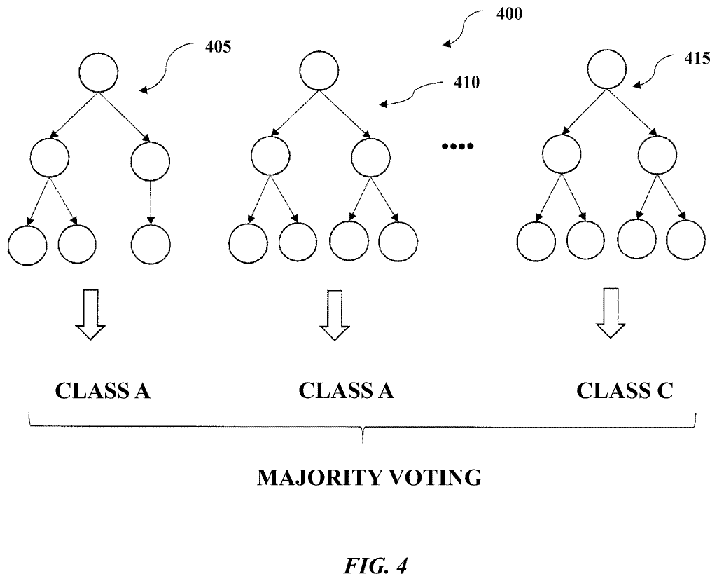

FIG. 4 illustrates a random forest 400. The random forest 400 (which may also be referred to as a random forest model) is an ensemble of decision trees 405, 410, and 415 with randomly selected features in each of the decision trees 405, 410, and 415 such that the random forest 400 can provide more stable and accurate outcomes. Outcomes may be determined by majority voting in the case of a classification problem. In the example of FIG. 4, the random forest 400, which has been trained previously by a training method, is used to decide between classifications A, B and C. For example, the random forest 400, with only the three decision trees shown in FIG. 4, would return the classification A by majority voting.

### Examples of Computing Systems

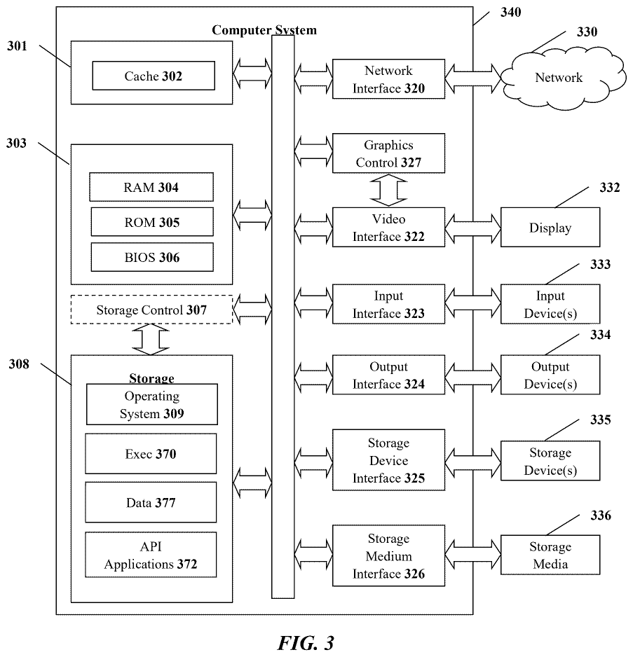

Referring to FIG. 3, a block diagram is shown depicting an example machine that includes a computer system 300 (e.g., a processing or computing system) within which a set of instructions can execute for causing a device to perform or execute any one or more of the aspects or methodologies for static code scheduling of the present disclosure. The components in FIG. 3 are examples and do not limit the scope of use or functionality of any hardware, software, embedded logic component, or a combination of two or more such components with particular implementations.

Computer system 300 may include one or more processors 301, a memory 303, and a storage 308 that communicate with each other, and with other components, via a bus 340. The bus 340 may also link a display 332, one or more input devices 333 (which may, for example, include a keypad, a keyboard, a mouse, a stylus, etc.), one or more output devices 334, one or more storage devices 335, and various tangible storage media 336. All of these elements may interface directly or via one or more interfaces or adaptors to the bus 340. For instance, the various tangible storage media 336 can interface with the bus 340 via storage medium interface 326. Computer system 300 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 300 includes one or more processor(s) 307 (e.g., central processing units (CPUs), general purpose graphics processing units (GPGPUs), or quantum processing units (QPUs)) that carry out functions. Processor(s) 301 optionally contains a cache memory unit 302 for temporary local storage of instructions, data, or computer addresses. Processor(s) 301 are configured to assist in execution of computer readable instructions. Computer system 300 may provide functionality for the components depicted in FIG. 3 as a result of the processor(s) 301 executing non-transitory, processor-executable instructions embodied in one or more tangible computer-readable storage media, such as memory 303, storage 308, storage devices 335, or storage medium 336. The computer-readable media may store software that implements particular operations, and processor(s) 301 may execute the software. Memory 303 may read the software from one or more other computer-readable media (such as mass storage device(s) 335, 336) or from one or more other sources through a suitable interface, such as network interface 320. The software may cause processor(s) 301 to carry out one or more processes or one or more operations of one or more processes described or illustrated herein. Carrying out such processes or operations may include defining data structures stored in memory 303 and modifying the data structures as directed by the software.

The memory 303 may include various components (e.g., machine readable media) including, but not limited to, a random access memory component (e.g., RAM 304) (e.g., static RAM (SRAM), dynamic RAM (DRAM), ferroelectric random access memory (FRAM), phase-change random access memory (PRAM), etc.), a read-only memory component (e.g., ROM 305), and any combinations thereof. ROM 305 may act to communicate data and instructions unidirectionally to processor(s) 301, and RAM 304 may act to communicate data and instructions bidirectionally with processor(s) 301. ROM 305 and RAM 304 may include any suitable tangible computer-readable media described below. In one example, a basic input/output system 306 (BIOS), including basic routines that help to transfer information between elements within computer system 300, such as during start-up, may be stored in the memory 303.

Fixed storage 308 is connected bidirectionally to processor(s) 301, optionally through storage control unit 307. Fixed storage 308 provides additional data storage capacity and may also include any suitable tangible computer-readable media described herein. Storage 308 may be used to store operating system 309, executable(s) 310, data 311, applications 312 (application programs), and the like. Storage 308 can also include an optical disk drive, a solid-state memory device (e.g., flash-based systems), or a combination of any of the above. Information in storage 308 may, in appropriate cases, be incorporated as virtual memory in memory 303.

In one example, storage device(s) 335 may be removably interfaced with computer system 300 (e.g., via an external port connector (not shown)) via a storage device interface 325. Particularly, storage device(s) 335 and an associated machine-readable medium may provide non-volatile or volatile storage of machine-readable instructions, data structures, program modules, or other data for the computer system 300. In one example, software may reside, completely or partially, within a machine-readable medium on storage device(s) 335. In another example, software may reside, completely or partially, within processor(s) 301.

Bus 340 connects a wide variety of subsystems. Herein, reference to a bus may encompass one or more digital signal lines serving a common function, where appropriate. Bus 340 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 300 may also include an input device 333. In one example, a user of computer system 300 may enter commands or other information into computer system 300 via input device(s) 333. Examples of an input device(s) 333 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some cases, the input device is a Kinect, Leap Motion, or the like. Input device(s) 333 may be interfaced to bus 340 via any of a variety of input interfaces 323 (e.g., input interface 323) including, but not limited to, serial, parallel, game port, USB, FIREWIRE, THUNDERBOLT, or any combination of the above.

In some cases, when computer system 300 is connected to network 330, computer system 300 may communicate with other devices, specifically mobile devices and enterprise systems, distributed computing systems, cloud storage systems, cloud computing systems, and the like, connected to network 330. Communications to and from computer system 300 may be sent through network interface 320. For example, network interface 320 may receive incoming communications (such as requests or responses from other devices) in the form of one or more packets (such as Internet Protocol (IP) packets) from network 330, and computer system 300 may store the incoming communications in memory 303 for processing. Computer system 300 may similarly store outgoing communications (such as requests or responses to other devices) in the form of one or more packets in memory 303 and communicated to network 330 from network interface 320. Processor(s) 301 may access these communication packets stored in memory 303 for processing.

Examples of the network interface 320 include, but are not limited to, a network interface card, a modem, and any combination thereof. Examples of a network 330 or network segment 330 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 330, may employ a wired or a wireless mode of communication. In general, any network topology may be used.

Information and data can be displayed through a display 332. Examples of a display 332 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 332 can interface to the processor(s) 301, memory 303, and fixed storage 308, as well as other devices, such as input device(s) 333, via the bus 340. The display 332 is linked to the bus 340 via a video interface 322, and transport of data between the display 332 and the bus 340 can be controlled via the graphics control 321. In some cases, the display is a video projector. In some cases, the display is a head-mounted display (HMD) such as a VR headset. In further cases, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further cases, the display is a combination of devices such as those disclosed herein.

In addition to a display 332, computer system 300 may include one or more other peripheral output devices 334 including, but not limited to, an audio speaker, a printer, a storage device, and any combinations thereof. Such peripheral output devices may be connected to the bus 340 via an output interface 324. Examples of an output interface 324 include, but are not limited to, a serial port, a parallel connection, a USB port, a FIREWIRE port, a THUNDERBOLT port, and any combinations thereof.

In addition or as an alternative, computer system 300 may provide functionality as a result of logic hardwired or otherwise embodied in a circuit, which may operate in place of or together with software to execute one or more processes or one or more operations of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Various illustrative logical blocks, modules, circuits, and algorithm operations described in connection with the examples disclosed herein may be implemented as electronic hardware, computer software, or combinations of both. To clearly illustrate this interchangeability of hardware and software, various illustrative components, blocks, modules, circuits, and operations have been described above generally in terms of their functionality.

The various illustrative logical blocks, modules, and circuits described in connection with the examples disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The operations of a method, a technique, or an algorithm described in connection with the examples disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium. An example storage medium may be coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Select televisions, video players, and digital music players with optional computer network connectivity may be suitable for use in the system described herein. Suitable tablet computers, in various cases, include those with booklet, slate, and convertible configurations.

In some cases, the computing device includes an operating system configured to perform executable instructions. The operating system is, for example, software, including programs and data, which manages the device's hardware and provides services for execution of applications. Suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems may include, by way of non-limiting examples, Microsoft® Windows®, Apple® Mac OS X®, UNIX®, and UNIX-like operating systems such as GNU/Linux®. In some cases, the operating system is provided by cloud computing. Suitable mobile smartphone operating systems may include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some cases, the systems, the methods, the computer-readable media, and the techniques disclosed herein include one or more non-transitory computer readable storage media encoded with a program including instructions executable by the operating system of an optionally networked computing device. In further cases, a computer readable storage medium is a tangible component of a computing device. In still further cases, a computer readable storage medium is optionally removable from a computing device. In some cases, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

In some cases, the systems, the methods, the computer-readable media, and the techniques disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. A computer program may be written in various versions of various languages.

The functionality of the computer readable instructions may be combined or distributed in various ways across various environments. In some cases, a computer program comprises one sequence of instructions. In some cases, a computer program comprises a plurality of sequences of instructions. In some cases, a computer program is provided from one location. In some cases, a computer program is provided from a plurality of locations. In some cases, a computer program includes one or more software modules. In some cases, a computer program includes, in part or in whole, one or more web applications, one or more mobile applications, one or more standalone applications, one or more web browser plug-ins, extensions, add-ins, or add-ons, or combinations thereof.

In some cases, a computer program includes a web application. A web application, in various cases, may utilize one or more software frameworks and one or more database systems. In some cases, a web application is created upon a software framework such as Microsoft®.NET or Ruby on Rails (RoR). In some cases, a web application utilizes one or more database systems including, by way of non-limiting examples, relational, non-relational, object oriented, associative, XML, and document oriented database systems. In further cases, suitable relational database systems include, by way of non-limiting examples, Microsoft® SQL Server, mySQL™, and Oracle®. A web application, in some cases, may be written in one or more versions of one or more languages. A web application may be written in one or more markup languages, presentation definition languages, client-side scripting languages, server-side coding languages, database query languages, or combinations thereof. In some cases, a web application is written to some extent in a markup language such as Hypertext Markup Language (HTML), Extensible Hypertext Markup Language (XHTML), or extensible Markup Language (XML). In some cases, a web application is written to some extent in a presentation definition language such as Cascading Style Sheets (CSS). In some cases, a web application is written to some extent in a client-side scripting language such as Asynchronous Javascript and XML (AJAX), Flash® ActionScript, JavaScript, or Silverlight®. In some cases, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some cases, a web application is written to some extent in a database query language such as Structured Query Language (SQL). In some cases, a web application integrates enterprise server products such as IBM® Lotus Domino®. In some cases, a web application includes a media player element. In some cases, a media player element utilizes one or more of many suitable multimedia technologies including, by way of non-limiting examples, Adobe® Flash®, HTML 5, Apple® QuickTime®, Microsoft® Silverlight®, Java™, and Unity®

In some cases, a computer program includes a mobile application provided to a mobile computing device. In some cases, the mobile application is provided to a mobile computing device at the time it is manufactured. In other cases, the mobile application is provided to a mobile computing device via the computer network described herein.

In view of the disclosure provided herein, a mobile application may be created using various hardware, languages, and development environments. In some cases, mobile applications are written in several languages. Suitable programming languages may include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and PhoneGap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums may be available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, and Samsung® Apps.

In some cases, a computer program includes a standalone application, which is a program that is run as an independent computer process, not an add-on to an existing process, e.g., not a plug-in. Standalone applications may be compiled. A compiler may be a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some cases, a computer program includes one or more executable complied applications.

In some cases, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Web browser plug-ins may include Adobe® Flash® Player, Microsoft® Silverlight®, and Apple® QuickTime®. In some cases, the toolbar comprises one or more web browser extensions, add-ins, or add-ons. In some cases, the toolbar comprises one or more explorer bars, tool bands, or desk bands.

Several plug-in frameworks may be available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some cases, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

In some cases, the systems, the methods, the computer-readable media, and the techniques disclosed herein include software, server, or database modules, or use of the same. Software modules may be created by techniques using machines, software, and languages. The software modules disclosed herein are implemented in a multitude of ways. In some cases, a software module comprises a file, a section of code, a programming object, a programming structure, a distributed computing resource, a cloud computing resource, or combinations thereof. In some cases, a software module comprises a plurality of files, a plurality of sections of code, a plurality of programming objects, a plurality of programming structures, a plurality of distributed computing resources, a plurality of cloud computing resources, or combinations thereof. In some cases, the one or more software modules comprise, by way of non-limiting examples, a web application, a mobile application, a standalone application, and a distributed or cloud computing application. In some cases, software modules are in one computer program or application. In some cases, software modules are in more than one computer program or application. In some cases, software modules are hosted on one machine. In some cases, software modules are hosted on more than one machine. In some cases, software modules are hosted on a distributed computing platform such as a cloud computing platform. In some cases, software modules are hosted on one or more machines in one location. In some cases, software modules are hosted on one or more machines in more than one location.

In some cases, the systems, the methods, the computer-readable media, and the techniques disclosed herein include one or more databases, or use of the same. In some cases, various databases may be suitable for storage and retrieval of one or more of (i) wearable data, (ii) responses to health queries, (iii) geographic data, etc., one or more of which may be historical, present, or future data or information. In some cases, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some cases, a database is Internet-based. In further cases, a database is web-based. In still further cases, a database is cloud computing-based. In a particular case, a database is a distributed database. In other cases, a database is based on one or more local computer storage devices.

While preferred embodiments of the present invention have been shown and described herein, it will be obvious to those skilled in the art that such embodiments are provided by way of example. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

As used in this specification and the appended claims, the terms “artificial intelligence,” “artificial intelligence techniques,” “artificial intelligence operation,” and “artificial intelligence algorithm” generally refer to any system or computational procedure that may take one or more actions to enhance or maximize a chance of achieving a goal. An example of such a goal is to mathematically or computationally model the probabilistic relationship between an input data (e.g., voice or biometric data) and an outcome like a mental condition detection (e.g., via generation of one or more scorecards). The term “artificial intelligence” may include “generative modeling,” “deep learning” (DL), “machine learning”, or “reinforcement learning” (RL). As used in this specification and the appended claims, the terms “machine learning,” “machine learning techniques,” “machine learning operation,” and “machine learning model” generally refer to any system or analytical or statistical procedure that may progressively improve computer performance of a task.

As used in this specification and the appended claims, “some embodiments,” “further embodiments,” or “a particular embodiment,” indicates that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

As used in this specification and the appended claims, when the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

As used in this specification and the appended claims, when the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in this specification, “or” is intended to mean an “inclusive or” or what is also referred to as a “logical OR,” wherein when used as a logic statement, the expression “A or B” is true if either A or B is true, or if both A and B are true, and when used as a list of elements, the expression “A, B, or C” is intended to include all combinations of the elements recited in the expression, for example, any of the elements selected from the group consisting of A, B, C, (A, B), (A, C), (B, C), and (A, B, C); and so on if additional elements are listed. As such, any reference to “or” herein is intended to encompass “and/or” unless otherwise stated.

As used in this specification and the appended claims, the indefinite articles “a” or “an,” and the corresponding associated definite articles “the” or “said,” are each intended to mean one or more unless otherwise stated, implied, or physically impossible. Yet further, it should be understood that the expressions “at least one of A and B, etc.,” “at least one of A or B, etc.,” “selected from A and B, etc.” and “selected from A or B, etc.” are each intended to mean either any recited element individually or any combination of two or more elements, for example, any of the elements from the group consisting of “A,” “B,” and “A AND B together,” etc.

As used in this specification and the appended claims “about” or “approximately” may mean within an acceptable error range for the value, which will depend in part on how the value is measured or determined, e.g., the limitations of the measurement system. For example, “about” may mean within 1 or more than 1 standard deviation. Alternatively, “about” may mean a range of up to 20%, up to 10%, up to 5%, or up to 1% of a given value. Where values are described in the application and claims, unless otherwise stated the term “about” meaning within an acceptable error range for the particular value may be assumed.

While preferred embodiments of the present invention have been shown and disclosed herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention disclosed herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

It should be noted that various illustrative or suggested ranges set forth herein are specific to their example embodiments and are not intended to limit the scope or range of disclosed technologies, but, again, merely provide example ranges for frequency, amplitudes, etc. associated with their respective embodiments or use cases. Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

It should be understood that, unless a term is expressly defined in this patent, there is no intent to limit the meaning of that term, either expressly or by implication, beyond its plain or ordinary meaning, and such term should not be interpreted to be limited in scope based at least in part on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are disclosed herein as including logic or a number of routines, subroutines, applications, or instructions. These may constitute either software (e.g., code embodied on a machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as disclosed herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, hardware modules may encompass a tangible entity, be that an entity that is physically constructed, permanently configured (e.g., hardwired), or temporarily configured (e.g., programmed) to operate in a certain manner or to perform certain operations disclosed herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and may operate on a resource (e.g., a collection of information). Elements that are described as being coupled and or connected may refer to two or more elements that may be (e.g., direct physical contact) or may not be (e.g., electrically connected, communicatively coupled, etc.) in direct contact with each other, but yet still cooperate or interact with each other.

The various operations of example methods disclosed herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines disclosed herein may be at least partially processor-implemented. For example, at least some of the operations of a method may be performed by one or more processors or processor-implemented hardware modules. The performance of certain operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

It will be understood that, although the terms first, second, etc. may be used herein to describe various elements, these elements should not be limited by these terms. These terms are only used to distinguish one element from another. For example, a first element may be termed a second element, and, similarly, a second element may be termed a first element, without departing from the scope of the present disclosure.

### EXAMPLES

### Example 1: Proof of Concept Study of 6,470 Positive Cases

A proof of concept (POC) study was performed to evaluate the analytical performance of the MIMI Variant Ranker. In the POC study, 6,740 cases from the testing and holdout datasets were tested by ranking variants with the MIMI Variant Ranker and observing the ranks of the diagnostic variant(s). The cases were scored based on the recall at k of the diagnostic variant(s) in the case. False negative (FN) cases were identified to be cases where the diagnostic variant(s) did not rank in the top k=3 (recall at 3<1.0).

Average recall at k is the mean recall at k across all tested cases (see Table 2) for case-averaged recall at k of the diagnostic variants. Over all 6,740 cases, the diagnostic variants were ranked in the top 3 by the MIMI Variant Ranker 98.3% of the time. In other words, the case average recall at 3 was 0.983.

The cases were subset in Table 2 by column per on the number of cat1 variants and genes reported per case: monoallelic refers to heterozygous or hemizygous calls for autosomal dominant disease cases or homozygous calls for autosomal recessive disease cases. Monoallelic cases have a single gene with diagnostic variants. The case average recall at 3 for monoallelic cases was 0.993.

The recall at k=3 for cat1 cases with diagnostic compound heterozygous variants was 0.891 (see column “compound heterozygous, rank of both variants”). A high-ranking single heterozygous call on an autosomal recessive-associated gene may signal to an analyst that the presence of a second weaker variant may be investigated. In this scenario, one of the two variants in the variant pair need to have a high rank. This is not different than our existing clinical workflow. Therefore, the POC study transformed the analysis such that only a single variant appears in the top k for a recall of 1.0; the recall at k=3 for that scenario is 0.981 (see column “compound heterozygous, rank of strongest variant”).

The recall at k=3 for complex disease cases (dual and triple genes with cat1 diagnoses) was 0.921. Strategies to identify “unexplained phenotypes” for the first diagnostic gene are outside the scope of this document.

TABLE 2
Case-averaged recall at k of diagnostic variants by case type.

| | | | Compound | Compound | |
|---|---|---|---|---|---|
| | | | Heterozy- | Heterozy- | |
| | | | gous, Rank | gous, Rank | Dual and |
| | | | of Strongest | of Both | Triple |
| k | All | Monoalle | Variant | Variants | Diagnoses |
| 3 | 0 | 0.99 | 0.981 | 0.891 | 0.921 |
| 5 | 0 | 0.99 | 0.988 | 0.953 | 0.955 |
| 10 | 0 | 0.99 | 1.000 | 0.982 | 0.974 |
| 15 | 0 | 0.99 | 1.000 | 0.990 | 0.981 |
| 20 | 0 | 0.99 | 1.000 | 0.996 | 0.984 |
| Case Count | 67 | 6015 | 571 | 571 | 154 |
| Percentage | 100% | 89% | 8.50% | 8.50% | 2.30% |
| of the | | | | | |
| Testing + | | | | | |
| [symbol] | | | | | |

[symbol]

indicates data missing or illegible when filed

The POC study investigated the results of the 41 FN monoallelic cases to identify issues with the MIMI Variant Ranker or flaws in the data that excuse the poor prioritization. Twenty-four cases were found to be valid FNs and seventeen cases were found to be excusable

In the valid FNs, the study includes one case where the diagnostic variant was the first diagnosis for that gene, so the Multiscore data is sparse. Single heterozygous calls in recessive genes pushed the diagnostic variant out of the top 3 ranks in nine cases, in three of those there were additional confounders: the diagnostic variant was in trans with a copy number variant (CNV) (out of scope at this time, n=1), the case was not a trio (n=1), Multiscore failure (the diagnostic gene had been observed six times internally and ranked poorly for a gene with that many SDC1 cases, n=1). One case is expected to difficult for the ranker but is still a true FN: The diagnostic variant was only reported after a Request to Review. The variant had a classification of likely benign (LBEN), a high frequency, and was found in a low complexity region per RepeatMatcher. This same variant was found to be in trans with a CNV. One other case failed due to the variant being in trans with a CNV and one other was not a trio. For the remaining 11 of the 24 FN cases, there is not a ready explanation.

There are number of interesting and actionable results found in the FNs. For single heterozygous variants, a challenge was identified with identification of nine FN cases in which the diagnostic variant would be in the top 3 but for single heterozygous variants on autosomal recessive genes that were prioritized higher. In solution, the machine learning model of the MIMI Variant Ranker includes validated disease gene inheritance feature data. A heterozygous variant on an autosomal recessive gene with no other heterozygous variant in trans would then be deprioritized.

Another interesting and actionable result found in the FNs applies to when cases are not a trio. In these cases, there is a challenge that two true FN cases were identified, plus four excusable FN cases that were duos or singletons. This indicates that the de novo status was asserted as false; while in reality, the de novo status is unknown. In solution, the machine learning model of the MIMI Variant Ranker includes the trio status of the case, with the assumption that the importance of the de novo case-level feature would then be deprioritized in non-trio cases.

Another interesting and actionable result found in the FNs applies to when there are out-of-scope variants in trans In these cases, there is a challenge that, for three identified FN cases, a CNV was in trans with a diagnostic variant seen as a single heterozygous call. In solution, the machine learning model of the MIMI Variant Ranker includes a feature indicating whether a CNV, short tandem repeat variants (STRVs), or other out-of-scope variant is in trans with a variant in trio cases, where phasing is possible. Those CNVs, STRVs, etc. may not be ranked by the MIMI Variant Ranker, but the feature described may prioritize the in-scope SNVs or INDELs that are in trans with such variants.

TABLE 3
Excusable false negatives (FNs) in monoallelic cases.

| Category/Reason | Count |
|---|---|
| Diagnostic variant is pushed out of the top 3 by artifacts. | 8 |
| Diagnostic variant is called as multiple events and a different | 4 |
| event was ranked in the top 3. | |
| The PATH variant classification was not migrated properly | 1 |
| between OSCAR and XA because of a manual nomenclature | |
| change. | |
| The de novo status of the diagnostic variant was not known until | 1 |
| a parent received targeted testing; therefore, the inheritance logic | |
| in Gregor failed to capture the de novo status. | |
| This variant was called by ExpansionHunter, and as an STRV, is | 2 |
| out of scope for MIMI Ranker. | |
| A second positive result was added later, so this is out of scope | 1 |
| for the monoallelic cases. The top ranked variant was the second | |
| diagnosis. | |

As shown in the POC study of Table 3, there are several FN cases where the poor performance of the MIMI Variant Ranker was excusable. In eight of these excusable cases, the diagnostic variant would be ranked in the top 3, but for artifacts captured by XA, especially the wildtype allele chrX:152864514C>CC that is highly prioritized in several cases. Gregor variant normalization solves nomenclature issues in XA, leading to errors like missed recognition of internal and GnomAD entries. Therefore, it is expected that GOR workflows will eliminate this issue. Artifacts were highly prioritized in some of the true FN cases, but removing the artifacts would not put the diagnostic variant in the top 3.

In another four of these excusable cases, the diagnostic variant was called as multiple events, and one or more of the events was ranked in the top three, but the event that was reported in XA was not in the top three. It is expected that GOR workflows will eliminate this issue. For example:
a. chr2:166229773CT> (rank 1)
b. chr2:166229771T>TA (rank 2)
c. chr2:166229773C>CAGG (rank 4, the reported variant in XA)

In one of these excusable cases, the diagnostic variant had been classified as PATH internally when the case was analyzed. However, the nomenclature had been changed manually. This prevented the correct migration of the variant data to the classified_variants table in XA, and the MIMI Variant Ranker data did not have the PATH information. It is expected that GOR workflows eliminates this issue by normalizing the nomenclature.

In another one of these excusable cases that was originally a duo, the de novo status of the diagnostic variant was not known until a parent received targeted testing. As a result, the Gregor inheritance logic would not be expected to capture the de novo status of the variant in this retrospective case.

In another two of these excusable cases, the diagnostic variant was an STRV, which is out of scope for the MIMI Variant Ranker. The study clarified the data cleaning process for future training and testing of the machine learning model of the MIMI Variant Ranker. In both cases, the diagnostic variant was chr19:46273461:C<CCAG.

In another one of these excusable cases, a second positive result was added after the case was closed, so this is out of scope for the monoallelic cases.

1.-22. (canceled)

23. A method for analyzing variants, comprising:
(a) performing whole genome sequencing or whole exome sequencing of a biological sample obtained or derived from a subject, thereby generating sequencing data;
(b) processing the sequencing data to determine a plurality of variants in said sequencing data, wherein the computer processing comprises formatting the sequencing data as a table-separated values (TSV) computer data file;
(c) transforming, by a computer, the TSV computer data file to a genomic ordered relational (GOR) computer data format, wherein the transforming comprises relationally ordering one or more columns of the TSV computer data file based on one or more parameters relating to at least a portion of the plurality of variants comprising: information relating to chromosomes, information relating to variant position, information relating to reference alleles, information relating to called alleles, or any combination thereof, thereby generating a GOR case variant computer data file;
(d) storing the GOR case variant computer data file in a computer memory;
(e) automatically annotating, by a computer at least a portion of the plurality of variants of the GOR case variant computer data file with one or more features comprising (i) variant-level features associated with the variant independently of characteristics of the subject and (ii) case-;
(f) transforming, by a computer, the stored GOR case variant computer data file to a list of case records formatted according to an Avro multi-level computer schema;
(g) transforming the list of case records to a serialized Avro file;
(h) prioritizing, utilizing a random forest or a neural network machine learning (ML) model, at least a subset of the annotated variants of the serialized Avro file, wherein the ML model is trained to perform operations comprising:
(1) receiving as input the serialized Avro file,
(2) determining a relevance with respect to a disease;
based at least in part on one or more features of the annotated variants, and
(3) prioritizing the at least portion of the plurality of variants based on the determined relevance with respect to the disease, thereby generating prioritized variants having a rank score in the serialized Avro file,
(i) transforming the prioritized variants from the Avro multi-level computer schema of the Avro file to the GOR computer data format, thereby ordering in the GOR format the prioritized variants based on the rank score; and
(j) selecting, using the one or more computer processors, a subset of the prioritized variants ordered in the GOR computer data format, wherein the selected subset of prioritized variants comprises a diagnostic variant most relevant to indicating a disease of the subject, wherein the selecting of the diagnostic variant has an accuracy rate of at least 90%.

24. The method of claim 23, wherein the variant-level features comprise variant population frequency or variant effects.

25. The method of claim 23, wherein the case-level features comprise variant segregation data or inheritance data and an association between the variant and one or more phenotypes associated with the subject.

26. The method of claim 25, wherein the phenotypes comprise disease phenotypes.

27. The method of claim 23, wherein the ML model comprises the random forest ML model.

28. The method of claim 23, wherein the ML model comprises the neural network ML model.

29.-32. (canceled)

33. The method of claim 23, wherein the plurality of variants comprises at least three thousand variants.

34. The method of claim 23, wherein the plurality of variants comprises at least ten thousand variants.

35. The method of claim 23, wherein the disease is a Mendelian disease.

36. The method of claim 23, wherein the disease is a suspected disease.

37. The method of claim 23, further comprising generating a training dataset for training the ML model.

38. The method of claim 37, further comprising querying, by the computer, at least a portion of the annotated variants of the GOR case variant computer data file.

39. The method of claim 38, further comprising receiving, by the computer in response to the querying, at least a subset of the portion of the annotated variants of the GOR case variant computer data file, thereby generating the training dataset.

40. The method of claim 39, wherein the at least subset of the portion of the annotated variants of the GOR case variant computer data file comprise annotations relating to clinical confirmation of the relevance of the at least subset of the portion of the annotated variants with respect to the disease.

41. The method of claim 40, further comprising generating a Disease Phenotype Resource (DiPR) case table comprising the clinical confirmation of the relevance of the at least subset of the portion of the annotated variants with respect to the disease.

42. The method of claim 41, further comprising querying the DiPR case table to generate the training dataset, wherein the training dataset comprises at least a subset of annotated variants of the DiPR case table.

43. The method of claim 41, further comprising updating the DiPR case table.

44. The method of claim 43, further comprising updating the DiPR case table with modified annotations of at least a subset of the annotated variants.

45. The method of claim 43, further comprising updating the DiPR case table by adding one or more annotated variants comprising annotations relating to the clinical confirmation of the relevance of the one or more annotated variants with respect to the disease.

46. The method of claim 40, further comprising utilizing the training dataset to retrain the ML model.
